(19) United States
(12) Patent Application Publication
(10) Pub. No.: US 2024/0197927 A1
(43) Pub. Date: Jun. 20, 2024

(54) COMBINATION COMPRISING A NEUROTENSIN RECEPTOR BINDING COMPOUND, GEMCITABINE AND NAB-PACLITAXEL

(71) Applicant: Fusion Pharmaceuticals Inc., Hamilton (CA)

(72) Inventors: Diane-Charlotte Imbs, Paris (FR); Elodie Lewkowicz, Paris (FR); Alexander McEwan, Toronto (CA); Thomas Rohban, Saint Germain En Laye (FR); Sylvie Rolland, Marolles en Hurepoix (FR); Daniel Stevens, Oxfordshire (GB); John R. Forbes, Burlington (CA)

(21) Appl. No.: 18/541,604

(22) Filed: Dec. 15, 2023

Related U.S. Application Data
(63) Continuation of application No. PCT/CA2022/050962, filed on Jun. 16, 2022.
(60) Provisional application No. 63/211,314, filed on Jun. 16, 2021.

(51) Int. Cl. A61K 51/04; A61K 31/7068; A61K 47/64; A61P 35/00

(52) U.S. Cl. CPC A61K 51/0482; A61K 31/7068; A61K 47/643; A61P 35/00

(57) ABSTRACT

The present invention relates to a combination comprising a neurotensin receptor binding compound, gemcitabine and nab-paclitaxel for use for the treatment of a neurotensin receptor overexpressing tumour in a subject.

### RELATED APPLICATION

This application is a continuation of International Application No. PCT/CA2022/050962 filed Jun. 16, 2022, which claims the benefit of and priority to U.S. Provisional Patent Application No. 63/211,314, filed on Jun. 16, 2021, the entire contents of which are hereby incorporated by reference for all purposes.

### BACKGROUND

The present invention relates to methods of treating tumours that overexpress neurotensin receptors, e.g. pancreatic cancer. In particular, the invention provides novel therapies based on the combination of a neurotensin receptor binding compound and chemotherapeutic agents, wherein said chemotherapeutic agents are gemcitabine and nab-paclitaxel.

Neurotensin NT is a 13 amino acid neuropeptide that is implicated in the regulation of luteinizing hormone and prolactin release and has significant interaction with the dopaminergic system.

Neurotensin binds to neurotensin receptors. Three neurotensin receptors are known, namely neurotensin receptor 1, also referred to as NTR1, neurotensin receptor 2, also referred to as NTR2, and neurotensin receptor 3, also referred to as NTR3. These neurotensin receptors are transmembrane receptors that bind the neurotransmitter neurotensin (Vincent et al., Trends Pharmacol. Sci., 1999, 20, 302-309; Pelaprat, Peptides, 2006, 27, 2476-2487). NTR1 and NTR2 which are encoded by the NTSR1 and NTSR2 genes, contain seven transmembrane helices and are G protein coupled. NTR3 has a single transmembrane domain and is encoded by the SORT1 gene.

The neurotensin receptor 1 (NTR1) is expressed predominantly in the central nervous system and intestine (smooth muscle, mucosa and nerve cells). Apart from the central nervous system, NTR1 is highly expressed in a mammalian body and a human body in particular on several neoplastic cells in several tumor indications, whereas the expression of NTR1 in most other tissues of the mammalian and the human body is either not existent or low. Under physiological conditions, weak or moderate expression of NTR1 is described only for colon.

The combination of the invention intends to treat the tumours that overexpress neurotensin receptors. By “overexpress”, it is meant a level of expression higher than in normal cells.

A “neurotensin overexpressing tumour” is also referred to as a “neurotensin positive tumour”, such as a “NTR1-positive tumour” or a “NTR1+ tumour”.

Binding and pharmacological studies demonstrate that neurotensin receptor 2 (NTR2) binds neurotensin as well as several other ligands already described for NTR1. However, unlike NTR1, NTR2 recognizes, with high affinity, levocabastine, a histamine H1 receptor antagonist previously shown to compete with neurotensin for low-affinity binding sites in the central nervous system.

The international application WO2014/086499 describes a table summarizing the expression of NTR1 as described in the prior art indicating the tissue, degree of expression, detection method and the respective references. This table is incorporated by reference.

The tumour indications where NTR1 is overexpressed include but are not limited to pancreatic ductal adenocarcinoma, small cell lung cancer, prostate cancer, colorectal cancer, breast cancer, meningioma, Ewing's sarcoma, pleural mesothelioma, head and neck cancer, non-small cell lung cancer, gastrointestinal stromal tumors, uterine leiomyoma and cutaneous T-cell lymphoma. A preferred group of NTR1 expressing tumor indications are pancreatic ductal adenocarcinoma, small cell lung cancer, prostate cancer, colorectal cancer, breast cancer, meningioma and Ewing's sarcoma.

Because of this selective expression of NTR1, NTR1 is regarded as a suitable target for therapeutic agents. Agonists and antagonists binding to NTR1 have been described in the prior art and WO2014/086499 describes an overview of such compounds.

WO2014/086499 also discloses a family of NTR1 antagonists suitable as diagnostic agents and/or pharmaceutical agents, particularly if conjugated to a diagnostic and/or therapeutic radionuclide.

Among the above-mentioned compounds, a first-in-human phase I study of ¹⁷⁷Lu-IPN01087 monotherapy (NCT03525392) is ongoing to assess preliminary safety and efficacy.

The combination of the invention intends to treat the tumours that overexpress neurotensin receptors, preferably pancreatic ductal adenocarcinoma and metastatic pancreatic ductal adenocarcinoma.

Pancreatic ductal adenocarcinoma (PDAC), which is the most common type of pancreatic cancer (95%), is a highly aggressive malignancy associated with a very poor clinical prognosis. With almost as many deaths (n=45,750) as new cases (n=56,770) in 2019, pancreatic cancer is currently the fourth leading cause of cancer death in the US, accounting for 7% of all cancer-related mortalities. The incidence and mortality rates of pancreatic cancer have been increasing for decades, and it is projected that pancreatic cancer will surpass liver, breast, prostate and colorectal cancers to become the second leading cause of cancer-related death by 2030. Currently, the Surveillance, Epidemiology, and End Results database reports the five-year survival rate for pancreatic cancer at 9.3%, which has improved only slightly in recent years, despite newer treatments. These statistics clearly highlight the need for new approaches to effectively treat this invariably fatal disease.

The poor prognosis of PDAC is mainly attributed to the lack of visible and specific symptoms and reliable biomarkers for early diagnosis, as well as an aggressive metastatic spread leading to poor response to treatments. According to the American Cancer Society (2019), more than half (52%) of patients with pancreatic cancer are diagnosed with metastatic disease. Furthermore, due to the broad heterogeneity of acquired genetic mutations and dense stromal environment, PDAC is highly resistant to current chemotherapy and radiotherapy regimens.

Since 1997, gemcitabine (CAS Registry No. 95058-81-4), a nucleoside metabolic inhibitor, has been the cornerstone of first-line treatment of patients with metastatic PDAC, after being shown to offer greater clinical benefit and a survival improvement over weekly 5-fluorouracil therapy. Gemcitabine is the active pharmaceutical ingredient in GEMZAR®.

Different gemcitabine-based combinations have since been intensely evaluated. However, only one combination showed anti-tumour activity and efficacy in metastatic PDAC first-line treatment: gemcitabine and nab-paclitaxel.

Nab-paclitaxel (CAS Registry No. 33069-62-4) is a nanoparticle albumin-bound formulation of paclitaxel (ABRAXANE®), which is a microtubule inhibitor. The regulatory approval in both the US and European Union of gemcitabine and nab-paclitaxel as a first-line therapy option for patients with metastatic PDAC was based on the findings of the MPACT phase III study, in which gemcitabine and nab-paclitaxel significantly improved overall survival (OS) (8.5 versus 6.7 months; p<0.001), progression-free survival (PFS) (5.5 versus 3.7 months; p<0.001), and overall response rate (ORR) (23% versus 7%; p<0.001), compared with gemcitabine alone.

Besides gemcitabine-based chemotherapy, FOLFIRINOX, a multiagent chemotherapy regimen composed of folinic acid (also known as leucovorin), fluorouracil (also known as 5-FU), irinotecan, and oxaliplatin, has emerged as an alternative treatment strategy for patients with metastatic PDAC showing a survival advantage (median OS of 11.1 versus 6.8 months; median PFS of 6.4 versus 3.3 months; and ORR of 31.6% versus 9.4%; p<0.001 for all parameters) compared to standard single-agent gemcitabine in a randomized, multi-center, phase II/III study.

However, despite the recent improvements in the treatment of PDAC, initially with demonstration of the activity of the FOLFIRINOX regimen and subsequently the approval of gemcitabine in combination with nab-paclitaxel, prognosis remains poor with median OS times of less than one year. Due to the difficulty in early detection, most patients with pancreatic cancer have advanced disease that has spread, or metastasized, to other parts of the body, thus limiting the number of patients who are able to seek surgical resection as a treatment option. To date, neither personalized medicine nor immunotherapy, which are considered the two recent revolutions of cancer treatment, have delivered major positive results in the treatment of PDAC. Reasons for the failure of most targeted therapies might be the complex genetic mechanisms taking place in pancreatic tumour cells, which favor resistance to cytotoxic as well as targeted agents, and the intricate tumour microenvironment that seems to protect the tumour through a discrete vessel network and a hypoxic milieu. Likewise, immunotherapy approaches may find difficulty in entering the stroma and reaching the tumour cells.

Hence, other therapeutic options should be sought to improve the outcome of patients with PDAC. In addition, considering the aggressive nature of the disease and the high resistance of PDAC to standard of care chemotherapy, a combination therapy approach is biologically justified.

Besides PDAC, NTR1 is also highly expressed in colorectal cancer.

Colorectal cancer is the third most common type of cancer, making up about 10% of all cases. Survival is directly related to detection and the type of cancer involved, but overall is poor for symptomatic cancers, as they are typically quite advanced. Survival rates for early-stage detection are about five times that of late-stage cancers.

There exists a need for more and different therapies that may prove to be effective in treating the above-mentioned tumours that overexpress neurotensin receptor, such as, but not only, pancreatic ductal adenocarcinoma.

Novel combinations of therapeutic agents to treat neurotensin receptor overexpressing tumours are presented herein.

### SUMMARY

The present invention provides a combination comprising a neurotensin receptor binding compound, gemcitabine and nab-paclitaxel for use for the treatment of a neurotensin receptor overexpressing tumour in a subject.

The invention also concerns a method for treating a neurotensin receptor overexpressing tumour in a subject, comprising administering to the subject an effective amount of a neurotensin receptor binding compound, gemcitabine and nab-paclitaxel.

The invention also concerns the use of a neurotensin receptor binding compound for the manufacture of a medicament for treating a neurotensin receptor overexpressing tumour in a subject, in combination with gemcitabine and nab-paclitaxel.

The invention also concerns a neurotensin receptor binding compound for use in treating a neurotensin receptor overexpressing tumour in a subject, wherein said neurotensin receptor binding compound is administered in combination with gemcitabine and nab-paclitaxel.

### DETAILED DESCRIPTION

The embodiments of the present disclosure relate to a combination comprising a neurotensin receptor binding compound, gemcitabine and nab-paclitaxel for use for the treatment of a neurotensin receptor overexpressing tumour in a subject.

### Neurotensin Receptor Binding Compound

According to a preferred embodiment, the neurotensin receptor binding compound is radiolabeled with a therapeutic radionuclide.

As used herein the term “radiolabeled” refers to a compound which is labelled with a radionuclide element, typically of metallic nature. Accordingly, a radiolabeled neurotensin receptor binding compound is a compound which comprises a radionuclide and which has specific binding affinity to neurotensin receptor. In some embodiments of the disclosure, said radiolabeled neurotensin receptor binding compound with specific binding affinity to at least NTR1 receptor.

More particularly, the neurotensin receptor binding compound comprises a neurotensin-targeting molecule linked to a chelating agent able to chelate the therapeutic radionuclide. Preferably, the chelating agent is covalently linked to the neurotensin-targeting molecule, either directly or via a linker.

As used herein, the term “neurotensin-targeting molecule” refers to a molecule with specific binding affinity to neurotensin receptor.

Preferably, the neurotensin receptor binding compound comprises a complex formed by a therapeutic radionuclide and a neurotensin-targeting molecule covalently linked to a chelating agent able to chelate the therapeutic radionuclide.

More preferably, the neurotensin receptor binding compound consists of a complex formed by a therapeutic radionuclide and a neurotensin-targeting molecule covalently linked to a chelating agent able to chelate the therapeutic radionuclide.

As used herein, the term “chelating agent” refers to an organic moiety comprising functional groups that are able to form non-covalent bonds with the radionuclide and, thereby, form stable radionuclide complex. Such chelating agents are either directly linked to the somatostatin receptor binding peptide or connected via a linker molecule, preferably it is directly linked. The linking bond(s) is (are) either covalent or non-covalent bond(s) between the cell receptor binding organic moiety (and the linker) and the chelating agent, preferably the bond(s) is (are) covalent.

The chelating agent can be selected from DOTA, NOTA, DTPA, DO3A, TETA, EDTA, NODAGA, NODASA, NOC, TRITA, CDTA, BAT, DFO, and HYNIC. The chelating agent is preferably DOTA.

Preferably, the neurotensin-targeting molecule is a neurotensin inhibitor, such as a neurotensin antagonist or a neurotensin agonist. More preferably, the neurotensin-targeting molecule is a NTR1 antagonist or a NTR1 agonist.

The therapeutic radionuclide is preferably selected from the beta-emitting radionuclides and the alpha-particle emitting radionuclides.

Beta-emitting radionuclides commonly used in cancer therapy comprise ¹⁷⁷Lu, ⁹⁰Y, ⁶⁷Cu, ¹³¹I, ¹⁸⁶Re, ¹⁸⁸Re, ²¹²Pb, and ²¹³Bi.

Alpha-particle emitting radionuclides commonly used in cancer therapy comprise ²¹¹At, ²¹²Pb, ²¹³Bi, ²²⁵Ac, and ²²⁷Th.

Preferably, the therapeutic radionuclide is selected from the group comprising ¹⁷⁷Lu, ⁹⁰Y, ⁶⁷Cu, ¹³¹I, ¹⁸⁶Re, ¹⁸⁸Re, ²¹¹At, ²¹²Pb, ²¹³Bi, ²²⁵Ac, and ²²⁷Th.

The therapeutic radionuclide is advantageously ¹⁷⁷Lu or ²²⁵Ac.

According to a preferred embodiment, the neurotensin receptor binding compound comprises a molecule of formula (i):

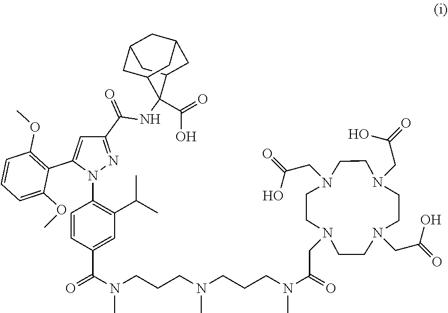 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=O)CN2CCN(CC(=O)O)CCN(CC(=O)O)CCN(CC(=O)O)CC2)cc1C(C)C]

or a complex thereof, preferably with a therapeutic radionuclide.

The compound of formula (i) is also known as IPN01087 or zalsenertant tetraxetan (CAS Registry No. 1613265-38-5).

The compound of formula (i) can be radiolabeled with all therapeutic radionuclides that can be chelated by DOTA chelator, such as ¹⁷⁷Lu, ⁹⁰Y, ⁶⁷Cu, and ²²⁵Ac. In some embodiments, the therapeutic radionuclide for chelating the compound of formula (i) is ¹⁷⁷Lu. In some embodiments, the therapeutic radionuclide for chelating the compound of formula (i) is ²²⁵Ac.

According to a preferred embodiment, the neurotensin receptor binding compound is a complex of formula (ii):

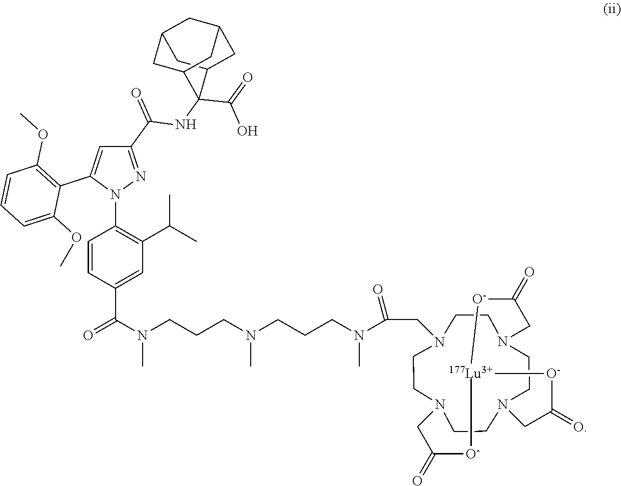

The compound of formula (i) is also known as ¹⁷⁷Lu-IPN01087, also referred to as ¹⁷⁷Lu-zalsenertant tetraxetan.

According to another embodiment, the neurotensin receptor binding compound is a compound of formula (I) as described in WO2014086499.

### Indication

The combination of the invention is useful for treating neurotensin receptor overexpressing tumours.

In particular, the neurotensin receptor overexpressing tumour can be selected in the group comprising pancreatic ductal adenocarcinoma (PDAC), small cell lung cancer, prostate cancer, colorectal cancer, breast cancer, meningioma, Ewing's sarcoma, pleural mesothelioma, head and neck cancer, non-small cell lung cancer, gastrointestinal stromal tumors, uterine leiomyoma and cutaneous T-cell lymphoma.

More particularly, the neurotensin receptor overexpressing tumour can be selected from pancreatic ductal adenocarcinoma and colorectal cancer. In some embodiments, the neurotensin receptor overexpressing tumour is pancreatic ductal adenocarcinoma. In some embodiments, the neurotensin receptor overexpressing tumour is colorectal cancer.

The combination of the invention is particularly useful for treating metastatic or unresectable pancreatic ductal adenocarcinoma.

According to an embodiment, the combination of the invention is particularly useful for treating neurotensin receptor overexpressing tumour resistant to treatment with gemcitabine and nab-paclitaxel.

According to an embodiment, the method of treatment of the invention is for treating subjects with metastatic PDAC who have not previously received therapy for pancreatic cancer and who demonstrated uptake of ¹⁷⁷Lu-IPN01087 or ¹¹¹In-IPN01087 in the tumour lesions.

According to a preferred embodiment, the neurotensin receptor binding compound is a NTR1 binding compound.

### Dosing Regimen

In the combination according to the invention, the neurotensin receptor binding compound is preferably for use in simultaneous, separate, or sequential combination with gemcitabine and nab-paclitaxel in the treatment of neurotensin receptor overexpressing tumour.

According to one embodiment, the neurotensin receptor binding compound is administered to the subject within about 5 minutes to within about 48 hours prior or after gemcitabine and nab-paclitaxel, preferably within about 24 hours after gemcitabine and nab-paclitaxel, more preferably within one hour after gemcitabine and nab-paclitaxel.

According to a preferred embodiment, the combination of the invention is administered to the subject according to a 28-day cycle, wherein the neurotensin receptor binding compound is administered on day 1 of the cycle and gemcitabine and nab-paclitaxel are administered on day 1, day 8 and day 15 of the cycle.

According to this embodiment, the combination of the invention is preferably administered for up to four 28-day cycles, for example for one 28-day cycle, two 28-day cycles, three 28-day cycles or four 28-day cycles. After a cycle, the next cycle starts on the next day after the end of said cycle: for example, the second cycle starts on the 29th day.

According to one embodiment, gemcitabine is administered at a dose of about 1000 mg/m² and nab-paclitaxel is administered at a dose of about 125 mg/m².

According to a preferred embodiment, the neurotensin receptor binding compound is radiolabeled with a therapeutic radionuclide and is more preferably the compound of formula (ii).

According to this embodiment, the radiolabeled neurotensin receptor binding compound is administered by injection IV at a dose of about 2 to 7 GBq per injection. As used herein, the term “injection IV” refers to an intravenous (IV) injection.

Preferably, the radiolabeled neurotensin receptor binding compound is administered at a dose of about 2 GBq per injection, at a dose of about 2.5 GBq per injection, at a dose of about 3 GBq per injection, at a dose of about 3.5 GBq per injection, at a dose of about 4 GBq per injection, at a dose of about 4.5 GBq per injection, at a dose of about 5 GBq per injection, at a dose of about 5.5 GBq per injection, at a dose of about 6 GBq per injection, at a dose of about 6.5 GBq per injection, at a dose of about 7 GBq per injection, or at a dose of about 7.5 GBq per injection.

According to a preferred embodiment, the combination of the invention is administered to the subject according to a 28-day cycle, wherein the radiolabeled neurotensin receptor binding compound is administered at a dose of 2 to 7 GBq by injection on day 1 of the cycle, nab-paclitaxel is administered at a dose of 125 mg/m² in a 30 to 40-minute intravenous infusion on day 1, day 8 and day 15 of the cycle, followed by gemcitabine at a dose of 1000 mg/m² in a 30-minute intravenous infusion on day 1, day 8 and day 15 of the cycle.

According to a preferred embodiment, the combination of the invention is administered to the subject according to a 28-day cycle, wherein:
on day 1 of the cycle, nab-paclitaxel is administered at a dose of 125 mg/m² in a 30-minute intravenous infusion, followed by gemcitabine at a dose of 1000 mg/m² in a 30-minute intravenous infusion, followed by the radiolabeled neurotensin receptor binding compound at a dose of 2 to 7 GBq by injection;
on day 8 (+1 day) of the cycle, nab-paclitaxel is administered at a dose of 125 mg/m² in a 30-minute intravenous infusion, followed by gemcitabine at a dose of 1000 mg/m² in a 30-minute intravenous infusion,
on day 15 (+1 day) of the cycle, nab-paclitaxel is administered at a dose of 125 mg/m² in a 30-minute intravenous infusion, followed by gemcitabine at a dose of 1000 mg/m² in a 30-minute intravenous infusion.

Preferably, on days 16 to 28 of the cycle, none of the three compounds of the combination is administered.

In some embodiments, the radiolabeled neurotensin receptor binding compound is administered as a unitary dosage of less than 40 MBq. In some embodiments, the radiolabeled neurotensin receptor binding compound is administered as a unitary dosage of 1-28 MBq (e.g., 3-25 MBq, 5-20 MBq, 5-15 MBq, or 10-15 MBq) to said subject. As used herein, “unitary dosage” typically refers to a single dose. To perform this invention, the radiolabeled neurotensin receptor binding compound can be administered as a unitary dosage for multiple times, i.e., administered multiple doses.

In some embodiments, the radiolabeled neurotensin receptor binding compound is the compound of formula (i) chelated with 225Ac. In some embodiments, the compound of formula (i) chelated with 225Ac is administered as a unitary dosage of about 5-15 MBq (e.g., about 5 MBq, about 6 MBq, about 7 MBq, about 8 MBq, about 9 MBq, about 10 MBq, about 11 MBq, about 12 MBq, about 13 MBq, about 14 MBq, or about 15 MBq).

### General Definitions

The use of the articles “a”, “an”, and “the” in both the description and claims are to be construed to cover both the singular and the plural, unless otherwise indicated herein or clearly contradicted by context. The terms “comprising”, “having”, “being of”, “including”, and “containing” are to be construed as open terms (i.e. meaning “including but not limited to”) unless otherwise noted. Additionally, whenever “comprising” or another open-ended term is used in an embodiment, it is to be understood that the same embodiment can be more narrowly claimed using the intermediate term “consisting essentially of” or the closed term “consisting of”.

The term “about” has herein the meaning that the following value may vary for ±20%, preferably ±10%, more preferably ±5%, even more preferably ±2%, even more preferably ±1%.

Unless otherwise defined, “%” has herein the meaning of weight percent (wt %), also referred to as weight by weight percent (w/w% ).

The phrase “treatment of” and “treating” includes the amelioration or cessation of a disease, disorder, or a symptom thereof. In particular, with reference to the treatment of a tumour, the term “treating” may refer to slowing or inhibiting the growth of the tumour, or the reducing the size of the tumour.

The terms “patient” and “subject” which are used interchangeably refer to a human being, including for example a subject that has cancer.

As used herein, the term “cancer” refers to cells having the capacity for autonomous growth, i.e., an abnormal state or condition characterized by rapidly proliferating cell growth. Hyperproliferative and neoplastic disease states may be categorized as pathologic, i.e. characterizing or constituting a disease state, or may be categorized as non-pathologic, i.e. a deviation from normal but not associated with a disease state. Unless specified otherwise, the term is meant to include all types of cancerous growths or oncogenic processes, metastatic tissues or malignantly transformed cells, tissues, or organs, irrespective of histopathologic type or stage of invasiveness.

“Combination” refers to a combined administration where the combination components of the present disclosure may be administered independently at the same time or separately within time intervals, especially where these time intervals allow that the combination components show a cooperative, e.g. synergistic effect. The single components may be packaged in a kit or separately. One or more of the components (e.g., powders or liquids) may be reconstituted or diluted to a desired dose prior to administration. The terms “co-administration” or “combined administration” or the like as utilized herein are meant to encompass administration of the selected combination components to a single subject in need thereof (e.g. a patient), and are intended to include treatment regimens in which the components are not necessarily administered by the same route of administration or at the same time.

As used herein, “simultaneous” or “simultaneously” is used to mean that the two agents are administered concurrently.

As used herein, “sequential, ” “sequentially,” “separate,” or “separately” is used to mean that the active agents are not administered concurrently, but one after the other. Thus, administration “sequential,” “sequentially,” “separate,” or “separately” may permit one agent to be administered within 5 minutes, 10 minutes or a matter of hours after the other provided the circulatory half-life of the first administered agent is such that they are both concurrently present in therapeutically effective amounts. The time delay between administrations of the components will vary depending on the exact nature of the components, the interaction there between, and their respective half-lives.

### BRIEF DESCRIPTION OF THE DRAWINGS

FIG. 1 represents the administration schedules from groups 1 to 9.

FIG. 2 represents the tumor volume of mice bearing subcutaneous HT-29 tumors from Groups 1, 2, 3 and 4 (Group 1=dots; Group 2=squares; Group 3=triangles; and Group 4=reversed triangles), wherein Group 2 was treated with a combination according to the invention.

FIG. 3 represents the tumor volume of mice bearing subcutaneous HT29 tumors from Groups 1, 3, 8 and 9 (Group 1=dots; Group 3=black triangles; Group 8=open triangles; Group 9=reversed triangles), wherein Group 8 was treated with a combination according to the invention.

FIG. 4 represents the mean tumor volume of mice bearing subcutaneous HT29 tumors from Groups 1, 3, 4 and 5 (Group 1=dots; Group 3=triangles; Group 4=reversed triangles; and Group 5=diamonds), wherein Group 5 was treated with a combination according to the invention.

FIG. 5 represents the mean tumor volume of mice bearing subcutaneous HT29 tumors from Groups 1, 3, 6 and 7 (Group 1=black dots; Group 3=triangles; Group 6=open dots; and Group 7=squares), wherein Group 6 was treated with a combination according to the invention.

FIG. 6 represents the administration schedules from groups 1E to 7E.

FIG. 7 represents the mean tumor volume of mice bearing subcutaneous AsPC1 tumors from Groups 1E, 3E, 4E and 5E (Group 1E=black triangles; Group 3E=white triangles; Group 4E=black squares; Group 5E=open squares), wherein Group 5E was treated with a combination according to the invention.

FIG. 8 represents the mean tumor volume of mice bearing subcutaneous AsPC1 tumors from Groups 1E, 3E, 6E and 7E (Group 1E=black triangles; Group 3E=open triangles; Group 6E=open squares; Group 7E=black squares), wherein Group 6E was treated with a combination according to the invention.

FIG. 9 represents the mean tumor volume of mice bearing subcutaneous AsPC1 tumors from Groups 1E, 2E, 3E and 7E (Group 1E=black triangles; Group 3E=open triangles; Group 2E=open squares; Group 7E=black squares), wherein Group 2E was treated with a combination according to the invention.

FIG. 10 represents the dosing regimen for a clinical trial aiming at testing the combination of the invention in human subjects.

### EXAMPLES

### Materials & Methods

### Reference Substances

Gemcitabine was dissolved in 0.9% NaCl solution to reach 20 and 30 mg/ml concentration. Abraxane was dissolved in 0.9% NaCl solution to reach 2.5 and 3.75 mg/mL final concentration.

### Radiolabeling of IPN01087 With Lutetium-177

The radiolabeling procedure was performed using ammonium acetate 0.4 M containing 0.325 M gentisic acid pH 4.2 buffer and lutetium-177 (¹⁷⁷LuCl₃, ITG, specific activity >3,000 GBq/mg at calibration).

In a microtube, lutetium-177 (¹⁷⁷LuCl₃) was mixed with ammonium acetate 0.4 M containing 0.325 M gentisic acid pH 4.2 buffer (2.8× volume of lutetium-177 solution) and IPN01087 to reach a specific activity of 85 MBq/μg. The reaction mixture was incubated at +85° C. for 30 minutes using a heating system. At the end of the incubation period, the radiolabeling incorporation was assessed by reversed phase liquid chromatography and thin layer chromatography. The radiolabeling mixture was then diluted with ammonium acetate 0.4 M containing 0.325 M gentisic acid pH 4.2 buffer, 0.9% NaCl solution, and DTPA Ca(Na₃) to reach the desired radioactive doses in MBq and concentrations.

### Vehicles

The vehicles of Gemcitabine and Abraxane are hereafter referred to as vehicle #1. The vehicle of ¹⁷⁷Lu-IPN01087 is hereafter referred to as vehicle #2.

### Example 1: Efficacy Study on Animals Induced With HT-29 Cells

### Cancer Cell Line

The HT-29 cell line was established from the primary tumor of a 44-year old Caucasian female patient with colon adenocarcinoma (Fogh J et al., J. Natl. Cancer Inst. 1977 Jul. 59(1): 221-26).

### Cell Culture Method

Tumor cells were grown as monolayer at 37° C. in a humidified atmosphere (5% CO2, 95% air). The culture medium was RPMI 1640 containing 2 mM L-glutamine supplemented with 10% fetal bovine serum. For experimental use, tumor cells were detached from the culture flask using accutase and neutralized by the addition of complete culture medium. The cells were counted, and viability exceeded 85% as assessed by 0.25% trypan blue exclusion assay. Two frozen pellets of HT29 tumor cells were prepared: one frozen cell pellet prepared during the in-vitro cell culture, and one frozen cell pellet prepared using the cell suspension used for tumor induction in mice.

### Animals

Healthy female Swiss Nude mice (Crl:NU(Ico)-Foxn1nu), 7 weeks old at reception, were obtained from Charles River.

### Tumor Induction

Tumors were induced by subcutaneous injection of 1×107 HT29 cells in 200 μL of RPMI 1640 medium containing matrigel (50:50, v:v, ref: 356237, BD Biosciences, France) into the right flank (in the axis of the heart) of 160 animals. HT29 tumor cell implantation was performed 24 hours after a whole-body irradiation with a gamma source (2 Gy (Nude mice), 60Co, BioMep, France). The day of tumor induction was considered as the day −11 (D-11).

### Randomization

Animals were grafted with subcutaneous HT-29 tumors on D-11 and randomized on day 0 (D0) based on their individual tumor volumes. On D0, mean tumor volumes between groups were 210 to 220 mm³, and individual tumor volumes ranged from 105 mm³ up to 315 mm³. Statistical analysis showed that there was no significant difference between groups at D-6, D-4, and D0.

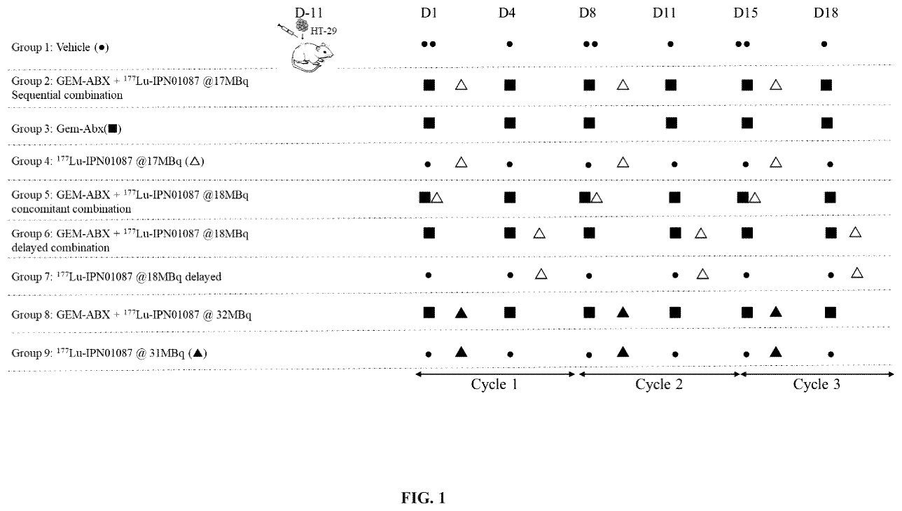

¹⁷⁷Lu-IPN01087 and the reference substances were administered by intravenous injection (IV) into the caudal vein via a catheter or by intraperitoneal (IP) injection. The recommended pH formulation for IV administration is pH 4.5-8.0 and for IP administration 4.5-8.0. The administration schedules are shown in FIG. 1. The animal groups were treated as follows:
Group 1 (control group): animals received twice weekly IV injection of 0.9% NaCl (vehicle #1) for a total of 6 injections combined with one weekly IV injection of a radiolabeling buffer/NaCl 0.9% solution mixture (vehicle #2) for a total of 3 injections.
Group 2 (“GEM-ABX+¹⁷⁷Lu-IPN01087 @17MBq” sequential combination groups): animals received twice weekly IV injection of Abraxane at 7.5 mg/kg for the first 3 injections and 5 mg/kg for the last 3 injections combined with twice weekly IV injection of Gemcitabine at 60 mg/kg for the first 3 injections and 40 mg/kg for the last 3 injections and sequentially combined with one weekly IV injection of ¹⁷⁷Lu IPN01087 at 17 MBq/mouse (0.38 nmol/mouse), starting 24 hours after the first Abraxane and Gemcitabine injection for 3 consecutive weeks.
Group 3 (“GEM-ABX” group): animals received twice weekly IV injection of Abraxane at 7.5 mg/kg for the first 3 injections and 5 mg/kg for the last 3 injections combined with twice weekly IV injection of Gemcitabine at 60 mg/kg for the first 3 injections and 40 mg/kg for the last 3 injections.
Group 4 (“¹⁷⁷Lu-IPN01087 @17MBq” group): animals received twice weekly IV injection of vehicle #1 for a total of 6 injections combined with one weekly IV injection of ¹⁷⁷Lu IPN01087 at 17 MBq/mouse (0.38 nmol/mouse) for 3 consecutive weeks.
Group 5 (“GEM-ABX+¹⁷⁷Lu-IPN01087 @18MBq” concomitant combination groups): animals received twice weekly IV injection of Abraxane at 7.5 mg/kg for the first 3 injections and 5 mg/kg for the last 3 injections combined with twice weekly IV injection of Gemcitabine at 60 mg/kg for the first 3 injections and 40 mg/kg for the last 3 injections and concomitantly combined with one weekly IV injection of ¹⁷⁷Lu IPN01087 at 18 MBq/mouse (0.40 nmol/mouse) at the first the first Abraxane and Gemcitabine injection for 3 consecutive weeks.
Group 6 (“GEM-ABX+¹⁷⁷Lu-IPN01087 @16MBq” delayed combination groups): animals received twice weekly IV injection of Abraxane at 7.5 mg/kg for the first 3 injections and 5 mg/kg for the last 3 injections (treatment schedule: TWx1,5) combined with twice weekly IV injection of Gemcitabine at 60 mg/kg for the first 3 injections and 40 mg/kg for the last 3 injections and combined with one weekly IV injection of ¹⁷⁷Lu IPN01087 at 16 MBq/mouse (0.36 nmol/mouse) starting 24 hours after the second injection of Gemcitabine and Abraxane for 3 consecutive weeks.
Group 7 (“¹⁷⁷Lu-IPN01087 @17MBq” groups): animals received twice weekly IV injection of vehicle #1 for a total of 6 injections combined with one weekly IV injection of ¹⁷⁷Lu IPN01087 at 17 MBq/mouse (0.38 nmol/mouse) starting 24 hours after the second injection of vehicle #1 for 3 consecutive weeks.
Group 8 (“GEM-ABX+¹⁷⁷Lu-IPN01087 @32MBq” sequential combination groups): animals received twice weekly IV injection of Abraxane at 7.5 mg/kg for the first 3 injections and 5 mg/kg for the last 3 injections combined with twice weekly IV injection of Gemcitabine at 60 mg/kg for the first 3 injections and 40 mg/kg for the last 3 injections and combined with one weekly IV injection of ¹⁷⁷Lu IPN01087 at 32 MBq/mouse (0.69 nmol/mouse) starting 24 hours after the first injection of Gemcitabine and Abraxane for 3 consecutive weeks.
Group 9 (“¹⁷⁷Lu-IPN01087 @31MBq” group): animals received twice weekly IV injection of vehicle #1 for a total of 6 injections combined with one weekly IV injection of ¹⁷⁷Lu IPN01087 at 31 MBq/mouse (0.68 nmol/mouse) starting 24 hours after the first injection of vehicle #1 for 3 consecutive weeks.

17MBq in mice is equivalent to about 4 GBq in human (human equivalent dose).

32MBq in mice is equivalent to about 7 GBq in human (human equivalent dose).

At the time of tumor relapse (or at termination if no escape occurs), the tumor from 3 out of 8 mice from each group were collected. Tumors will be weighed, flash-frozen, and then stored at −80° C.

The treatment efficacy was assessed in terms of the effects of the treatments on the tumor volumes of treated animals relative to control animals. The tumor volume was estimated by the formula:

$$Tumor Volume = \frac{{width}^{2} \times length}{2}$$

Tumors that were palpable and not measurable using calipers were assigned a volume of 4 mm³, indicating the technical limit measure. Tumor volume of 1000 mm³ were considered to be equal to 1 g. Individual, mean, and median tumor volumes were measured.

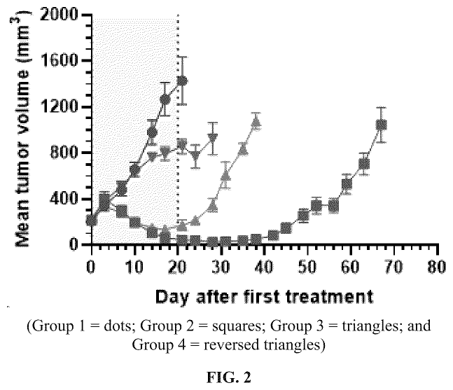

Results are presented in FIGS. 2 to 5.

FIG. 2 shows HT-29 tumor growth in mice treated with vehicles (Group 1=dots), Gemcitabine/Abraxane (Group 3=triangles), ¹⁷⁷Lu-IPN01087 (Group 4=reversed triangles), or a sequential combination of Gemcitabine/Abraxane and ¹⁷⁷Lu-IPN01087 (Group 2=squares). Vertical dotted lines indicate the end of the treatments. While Gemcitabine/Abraxane or ¹⁷⁷Lu-IPN01087 treatments produced an inhibitory effect on tumor growth as compared to the vehicles, the sequential combination of Gemcitabine/Abraxane and ¹⁷⁷Lu-IPN01087 caused a greater tumor volume regression during the treatment period. The regression was stabilized up to Day-42 before relapse.

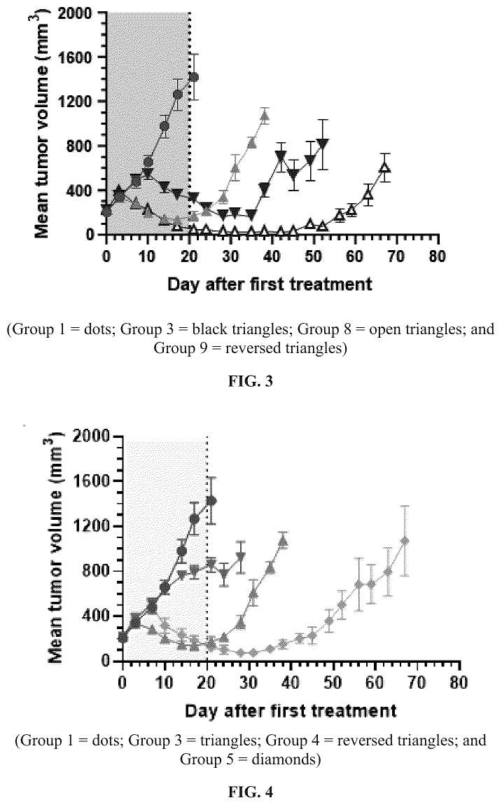

Similarly, the data shown in FIG. 3 demonstrate that a sequential combination of Gemcitabine/Abraxane and ¹⁷⁷Lu-IPN01087 at a higher dose of 31 MBq (Group 8=open triangles) produced a greater and sustained regression of the tumor. The tumor regression was followed by a stabilization up to Day-52 before relapse.

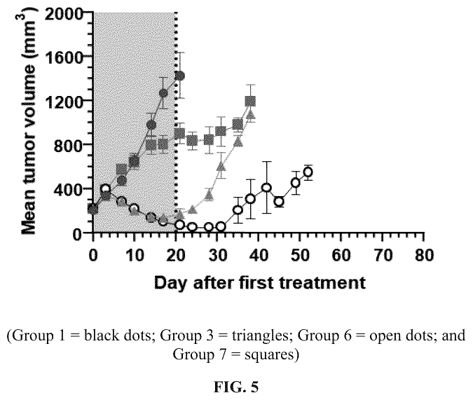

FIG. 4 and FIG. 5 show the data obtained from the mice treated with a concomitant combination of Gemcitabine/Abraxane and ¹⁷⁷Lu-IPN01087 (Group 5=diamonds) or a delayed sequential combination of Gemcitabine/Abraxane and ¹⁷⁷Lu-IPN01087 (Group 6=open dots). Both combinations caused a sustained tumor regression when compared with the single agent treatments.

### Example 2: Efficacy Study on Animals Induced With AsPC-1 Cells

### Cancer Cell Line

The AsPC 1 cell line was isolated from a metastatic site (ascites) from a 62-year old female patient (Chen W H. et al., In Vitro. 1982 Jan; 18(1):24-34).

### Cell Culture Method

Tumor cells were grown as monolayer at 37° C. in a humidified atmosphere (5% CO2, 95% air). The culture medium was RPMI 1640 containing 2 mM L-glutamine supplemented with 10% fetal bovine serum. For experimental use, tumor cells were detached from the culture flask using accutase and neutralized by the addition of complete culture medium. The cells were counted, and viability exceeded 85% as assessed by 0.25% trypan blue exclusion assay. Two frozen pellets of HT29 tumor cells were prepared: one frozen cell pellet prepared during the in-vitro cell culture, and one frozen cell pellet prepared using the cell suspension used for tumor induction in mice

### Animals

Healthy female Swiss Nude mice (Crl:NU(Ico)-Foxn1^{nu}), 6 weeks old at reception, were obtained from Charles River.

### Tumor Induction

Tumors were induced by subcutaneous injection of 1×107 AsPC 1 cells in 200 μL of RPMI 1640 medium containing matrigel (50:50, v:v, ref: 356237, BD Biosciences, France) into the right flank (in the axis of the heart) of 150 animals. AsPC 1 tumor cell implantation was performed 72 hours after whole-body irradiation with a gamma source (2 Gy (Nude mice), 60Co, BioMep, France).

### Randomization

Animals were randomized by individual tumor volume when tumors reach a mean volume of 150-200 mm3. Sixty-four animals (64) out of 150 were randomized into eight groups of eight animals, each using Vivo Manager® software (Biosystemes, France). Homogeneity between groups were tested by an analysis of variance (ANOVA).

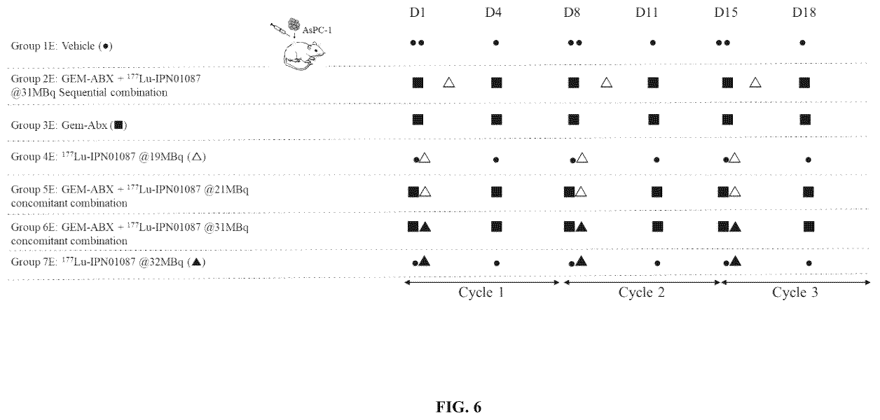

¹⁷⁷Lu-IPN01087 and the reference substances were administered by intravenous injection (IV) into the caudal vein via a catheter. The recommended pH formulation for IV administration is pH 4.5-8.0. The administration schedules are shown in FIG. 6. The animal groups were treated as follows:
Group 1E (control group): animals received twice weekly IV injection of 0.9% NaCl (vehicle #1) for a total of 6 injections combined with one weekly IV injection of a radiolabeling buffer/NaCl 0.9% solution mixture (vehicle #2) for a total of 3 injections
Group 2E (“GEM-ABX+¹⁷⁷Lu-IPN01087 @31MBq” sequential combination groups): animals received twice weekly IV injection of Abraxane at 6.25 mg/kg for the first 3 injections and 5 mg/kg for the last 3 injections combined with twice weekly IV injection of Gemcitabine at 50 mg/kg for the first 3 injections and 40 mg/kg for the last 3 injections and sequentially combined with one weekly IV injection of ¹⁷⁷Lu IPN01087 at 31 MBq/mouse (0.68 nmol/mouse), starting 24 hours after the first Abraxane and Gemcitabine injection for 3 consecutive weeks.
Group 3E (“GEM-ABX” group): animals received twice weekly IV injection of Abraxane at 6.25 mg/kg for the first 3 injections and 5 mg/kg for the last 3 injections combined with twice weekly IV injection of Gemcitabine at 50 mg/kg for the first 3 injections and 40 mg/kg for the last 3 injections.
Group 4E (“¹⁷⁷Lu-IPN01087 @19MBq” group): animals received twice weekly IV injection of vehicle #1 for a total of 6 injections combined with one weekly IV injection of ¹⁷⁷Lu IPN01087 at 19MBq/mouse (0.43 nmol/mouse) for 3 consecutive weeks.
Group 5E (“GEM-ABX+¹⁷⁷Lu-IPN01087 @21MBq” concomitant combination groups): animals received twice weekly IV injection of Abraxane at 6.25 mg/kg for the first 3 injections and 5 mg/kg for the last 3 injections combined with twice weekly IV injection of Gemcitabine at 50 mg/kg for the first 3 injections and 40 mg/kg for the last 3 injections and concomitantly combined with one weekly IV injection of ¹⁷⁷Lu IPN01087 at 21MBq/mouse (0.45 nmol/mouse) at the first Abraxane and Gemcitabine injection for 3 consecutive weeks.
Group 6E (“GEM-ABX+¹⁷⁷Lu-IPN01087 @31MBq” concomitant combination groups): animals received twice weekly IV injection of Abraxane at 6.25 mg/kg for the first 3 injections and 5 mg/kg for the last 3 injections combined with twice weekly IV injection of Gemcitabine at 50 mg/kg for the first 3 injections and 40 mg/kg for the last 3 injections and concomitantly combined with one weekly IV injection of ¹⁷⁷Lu IPN01087 at 31MBq/mouse (0.7 nmol/mouse) for 3 consecutive weeks.).
Group 7E (“¹⁷⁷Lu-IPN01087 @32MBq” group): animals received twice weekly IV injection of vehicle #1 for a total of 6 injections combined with one weekly IV injection of ¹⁷⁷Lu IPN01087 at 32MBq/mouse (0.71 nmol/mouse) for 3 consecutive weeks.

At the time of tumor relapse (or at termination if no escape occurs), the tumor from 3 out of 8 mice from each group were collected. Tumors will be weighed, flash-frozen, and then stored at −80° C.

The treatment efficacy was assessed in terms of the effects of the treatments on the tumor volumes of treated animals relative to control animals. The tumor volume was estimated by the formula:

$$Tumor Volume = \frac{{width}^{2} \times length}{2}$$

Tumors which were palpable and not measurable using calipers were assigned a volume of 4 mm³, indicating the technical limit measure. Tumor volume of 1000 mm³ were considered to be equal to 1 g. Individual, mean and median tumor volumes were measured.

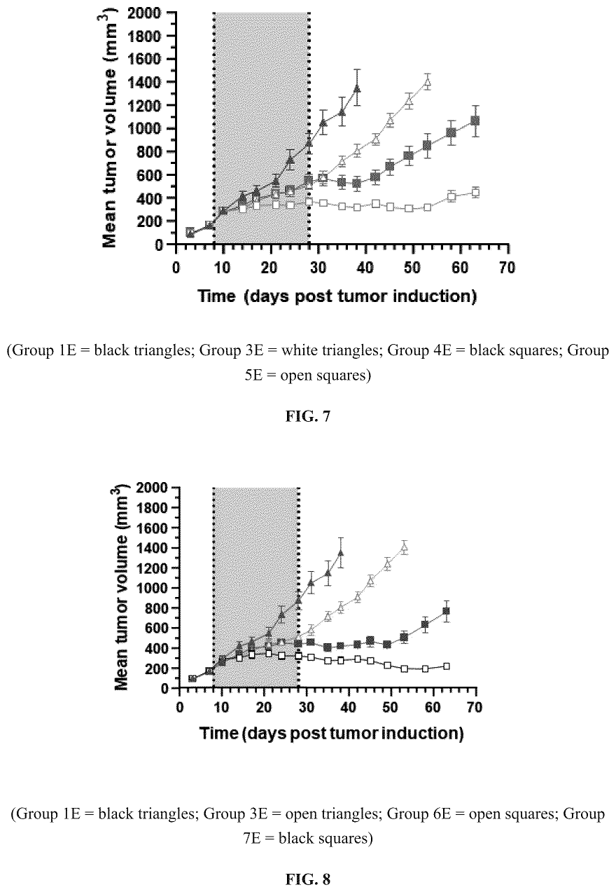

Results are presented in FIGS. 7 to 9.

FIG. 7 shows AsPC-1 tumor growth in mice treated with vehicles (Group 1E=black triangles), Gemcitabine/Abraxane (Group 3E=open triangles), ¹⁷⁷Lu-IPN01087 (Group 4E=black squares), or a concomitant combination of Gemcitabine/Abraxane and ¹⁷⁷Lu-IPN01087 (Group 5E=open squares). Vertical dotted lines indicate the start and the end of treatments. While Gemcitabine/Abraxane and ¹⁷⁷Lu-IPN01087 treatment alone produced an antitumor effect when compared to the vehicles, the concomitant combination of Gemcitabine/Abraxane and ¹⁷⁷Lu-IPN01087 significantly improved tumor growth control as compared to either of the single treatment.

Similarly, a concomitant combination of Gemcitabine/Abraxane and ¹⁷⁷Lu-IPN01087 at a higher dose of 32MBq (Group 6E=open squares) displayed better AsPC-1 tumor growth control as compared to Gemcitabine/Abraxane (Group 3E=open triangles) and ¹⁷⁷Lu-IPN01087 (Group 7E=black squares) alone (FIG. 8).

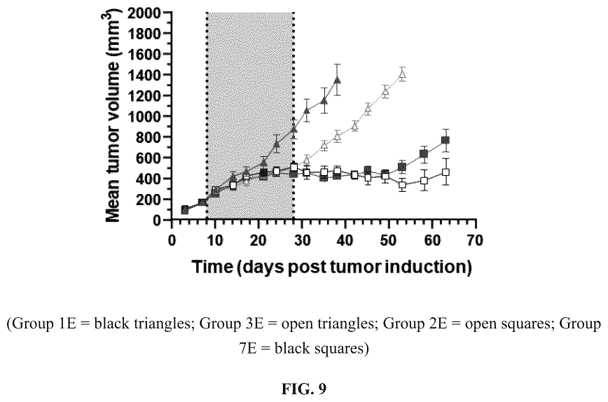

In addition, the data shown in FIG. 9 demonstrate that a sequential combination of Gemcitabine/Abraxane and ¹⁷⁷Lu-IPN01087 at a higher dose of 31 MBq (Group 2E=open squares) displayed a better tumor growth control than Gemcitabine/Abraxane (Groupe 3E=open triangles) and ¹⁷⁷Lu-IPN01087 (Group 7E=black squares) alone.

Overall, in combination with gemcitabine and nab-paclitaxel chemotherapy regimen, the ¹⁷⁷Lu-IPN01087 radiotherapy showed improved tumor growth control and even prolonged regression in the and HT-29 colon and AsPC-1 pancreatic cancer models, respectively, compared to the chemotherapy or radiotherapy regimens alone.

### Toxicity

Weight losses were observed in all groups of mice. The mean weight losses observed in the groups receiving the combination treatment (−6% to −17%; Group 2E, 5E, and 6E) were similar to those observed in the groups receiving either Gemcitabine/Abraxane (−8% to −11%; Groupe 3E) or ¹⁷⁷Lu-IPN01087 (−4% to −10%, Groups 4E, and 7E) alone.

### Example 3: Clinical Trial

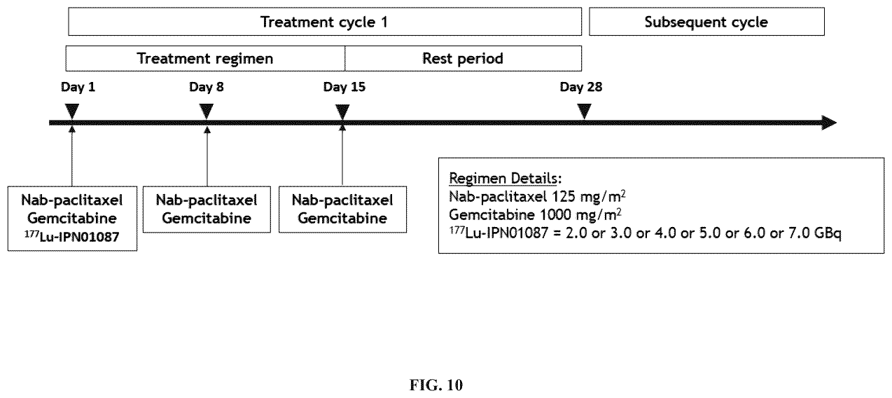

The combination of the invention will be administered during a cycle of 28 days as follows (FIG. 10):
on day 1 of each cycle: nab-paclitaxel 125 mg/m² in a 30-40 minute intravenous (i.v.) infusion, followed by gemcitabine 1000 mg/m² in a 30-minute i.v. infusion, followed by ¹⁷⁷Lu-IPN01087 injection at a dose predefined in each dose escalation cohort (2 GBq to 7 GBq),
on day 8 (+1 day) of each cycle: nab-paclitaxel 125 mg/m² in a 30-40 minute i.v. infusion, followed by gemcitabine 1000 mg/m² in a 30-minute infusion,
on day 15 (+1 day) of each cycle: nab-paclitaxel 125 mg/m² in a 30-40 minute i.v. infusion, followed by gemcitabine 1000 mg/m² in a 30-minute infusion, and
on days 16 to 28 of each cycle: none of the three compounds will be administered (rest period).

Gemcitabine and nab-paclitaxel will be administered according to the SmPC or US Prescribing Information (USPI) for metastatic pancreatic adenocarcinoma: the RD of nab-paclitaxel in combination with gemcitabine is 125 mg/m² administered i.v. over 30 minutes on days 1, 8 and 15 of each 28-day cycle. The concurrent RD of gemcitabine is 1000 mg/m² administered i.v. over 30 minutes immediately after the completion of nab-paclitaxel administration on days 1, 8 and 15 of each 28-day cycle. Dose reduction and modifications of both gemcitabine and nab-paclitaxel will be performed according to the SmPC and USPI: “Dose adjustments during treatment of pancreatic adenocarcinoma”.

Up to 6 dose levels of ¹⁷⁷Lu-IPN01087 are planned to be tested: 2.0 GBq, 3.0 GBq, 4.0 GBq, 5.0 GBq, 6.0 GBq, 7.0 GBq.

1. A method of treating a neurotensin receptor overexpressing tumor, comprising administering to a subject in need thereof a therapeutically effective amount of a combination comprising a neurotensin receptor binding compound, gemcitabine and nab-paclitaxel.

2. The method of claim 1, wherein the neurotensin receptor binding compound is radiolabeled with a therapeutic radionuclide.

3. The method of claim 2, wherein the therapeutic radionuclide is selected from the group comprising consisting of ¹⁷⁷Lu, ⁹⁰Y, ⁶⁷Cu, ¹³¹I, ¹⁸⁶Re, ¹⁸⁸Re, ²¹¹ At, ²¹²Pb, ²¹³Bi, ²²⁵ Ac, and ²²⁷Th.

4. The method of claim 2, wherein the therapeutic radionuclide is ¹⁷⁷Lu or ²²⁵Ac.

5. The method of claim 1, wherein the neurotensin receptor binding compound comprises a molecule of formula (i): 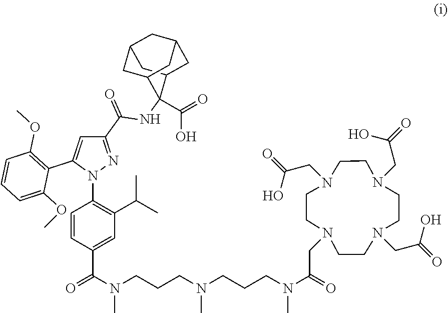 [SMILES: COc1cccc(OC)c1-c1cc(C(=O)NC2(C(=O)O)C3CC4CC(C3)CC2C4)nn1-c1ccc(C(=O)N(C)CCCN(C)CCCN(C)C(=O)CN2CCN(CC(=O)O)CCN(CC(=O)O)CCN(CC(=O)O)CC2)cc1C(C)C]
or a complex thereof.

6. The method of claim 1, wherein the neurotensin receptor binding compound is radiolabeled with ²²⁵Ac.

7. The method of claim 1, wherein the neurotensin receptor binding compound is a complex of formula (ii):

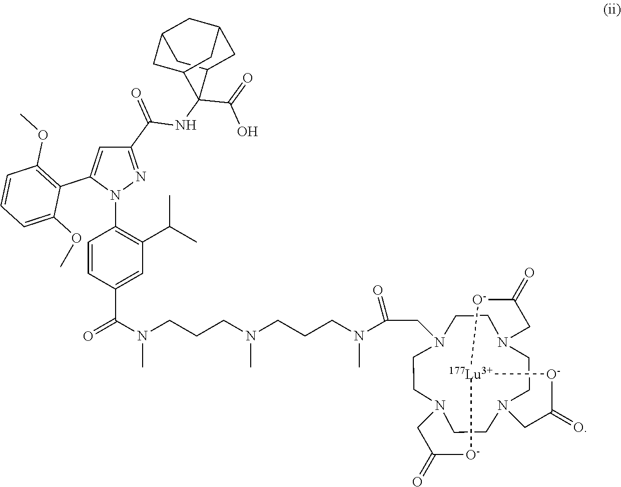

8. The method of claim 1, wherein the neurotensin receptor overexpressing tumour is selected in the group comprising pancreatic ductal adenocarcinoma and colorectal cancer.

9. The method of claim 1, wherein the neurotensin receptor binding compound is a NTR1 binding compound.

10. The method of claim 1, wherein the neurotensin receptor binding compound is administered in simultaneous, separate, or sequential combination with gemcitabine and nab-paclitaxel.

11. The method of claim 1, characterized in that the combination is administered to the subject according to a 28-day cycle, wherein the neurotensin receptor binding compound is administered on day 1 of the cycle and gemcitabine and nab-paclitaxel are administered on day 1, day 8 and day 15 of the cycle.

12. The method of claim 11, characterized in that the combination is administered for up to four 28-day cycles.

13. The method of claim 11, wherein gemcitabine is administered at a dose of about 1000 mg/m² and nab-paclitaxel is administered at a dose of about 125 mg/m².

14. The method of claim 1, wherein the radiolabeled neurotensin receptor binding compound is administered by injection IV at a dose of about 2 to 7 GBq per injection.

15. The method of claim 1, characterized in that the combination is administered to the subject according to a 28-day cycle, wherein:
on day 1 of the cycle, nab-paclitaxel is administered at a dose of 125 mg/m² in a 30 minute intravenous infusion, followed by gemcitabine at a dose of 1000 mg/m² in a 30-minute intravenous infusion, followed by the radiolabeled neurotensin receptor binding compound at a dose of 2 to 7 GBq by injection;
on day 8 (±1 day) of the cycle, nab-paclitaxel is administered at a dose of 125 mg/m² in a 30-minute intravenous infusion, followed by gemcitabine at a dose of 1000 mg/m² in a 30-minute intravenous infusion, and
on day 15 (±1 day) of the cycle, nab-paclitaxel is administered at a dose of 125 mg/m² in a 30 minute intravenous infusion, followed by gemcitabine at a dose of 1000 mg/m² in a 30-minute intravenous infusion.

16. The method of claim 15, wherein on days 16 to 28 of the cycle, none of the three compounds of the combination is administered.

17. The method of claim 1, wherein the radiolabeled neurotensin receptor binding compound is administered as a unitary dosage of less than 40 MBq.
